(19) United States
(12) Patent Application Publication
(10) Pub. No.: US 2007/0166317 A1
(43) Pub. Date: Jul. 19, 2007

(54) Microparticles

(76) Inventor: Gavin William Halbert, Glasgow (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/569,650

(22) PCT Filed: Aug. 27, 2004
(86) PCT No.: PCT/GB04/03679

§ 371 Date: Feb. 5, 2007

(30) Foreign Application Priority Data
Aug. 29, 2003 (GB) 0320268.6

(51) Int. Cl. A61K 39/00; A01N 25/12; A61K 9/14

(52) U.S. Cl. 424/184.1; 424/489; 977/906; 504/367

(57) ABSTRACT

The present invention relates to microparticles formed from polymer materials and comprising a peptide anchored thereto for binding to a cell surface receptor, for delivering agents to cells and to a method of making such microparticles.

### FIELD OF THE INVENTION

The present invention relates to microparticles formed from polymer materials and comprising a peptide anchored thereto for binding to a cell surface receptor, for delivering agents to cells and to a method of making such microparticles.

### BACKGROUND TO THE INVENTION

There is a continuing need to deliver agents to cells. To this end many forms of delivery agents including polymer microparticles have been formed. Nevertheless, there remains a need for microparticles which can target and deliver agents to specific cell types.

It is an object of the present invention to provide microparticles which can target specific cell types by causing the microparticles to bind to a receptor on the surface of the cell.

### SUMMARY OF THE INVENTION

Generally speaking the present invention is based on observations by the present inventors that peptides comprising cell receptor binding sequences can be modified so as to include a hydrophobic moiety, such that the peptides can be anchored to the surface of polymer microparticles. Such modified microparticles can thereafter be used to target agents to cells.

Thus, in a first aspect the present invention provides a microparticle for use in delivering an agent or agents to a cell, the microparticle comprising:
a) a polymer shell;
b) an agent or agents for delivery to a cell; and
c) a peptide component comprising a hydrophobic moiety wherein the hydrophobic moiety is capable of anchoring the peptide to the polymer shell and the peptide is intended to target the microparticle to a receptor on the surface of the cell.

The polymer shell may be made from any suitable polymer and may, for example, be biodegradable and/or biocompatible. The term “shell” is understood to relate to in general an object with a hollow core. Generally the microparticles are spherical or spheroid in nature. Suitable polymers for producing the microparticles of the present invention include polyesters such as polylactide, polyglycolide, copolymers of lactide and glycolide, polyhydroxybutyrate, polycaprolactone, copolymers of lactic acid and lactone, copolymers of lactic acid and PEG, copolymers of a-hydroxy acids and α-amino acids(polydepsipeptides), polyanhydrides, polyorthoesters, polyphosphazenes, copolymers of hydroxybutyrate and hydroxyvalerate, poly(ethylene carbonate), copoly(ethylene carbonate), polyethylene terephthalate, polystyrene/latex polymers, or mixtures of these polymers.

The microparticles preferably have a size in the range 10 nm to 200 μm.

The term “agent” as used herein includes any agent which it may be desired to administer to a human or animal body for any purpose, including therapeutic, pharmaceutical, pharmacological, diagnostic, cosmetic and prophylactic agents. The term is also used to include any agents which it may be desired to administer to plants by controlled release, such as agrochemicals including herbicides, pesticides and fertilizers.

The agent may be a pharmaceutical, a polypeptide, peptide or protein, a carbohydrate or a nucleic acid such as DNA.

The agent may also be an antigen for use in vaccines and these include polypeptides, proteins, glycoproteins that are obtained from bacterial, viral and parasitic sources or produced by synthetic methods. The term “antigen” is used herein to include any material which will cause an antibody reaction of any sort when administered. Such antigens can be administered by injection or by delivery to various mucosal sites (nasal, oral, vaginal, rectal, colonic).

Vaccines for the treatment of allergens and for auto immune diseases are well described in the prior art. For example in autoimmune disease it has been suggested that the slow administration of essential factors can be beneficial. Such factors can include insulin for the treatment of diabetes and collagen for treating rheumatoid arthritis.

The microparticles may be used to deliver the agent to cells which are in vitro or in vivo. The microparticles are useful for delivering a wide range of agents and can be administered by a wide range of routes, depending on the agent to be delivered. The microparticles may be adapted for injection, either intramuscularly, intravenously, subcutaneously, intraarticularly or intraperitoneally. The microparticles may be adapted for administration to the dermal or epidermal layer of the skin by injection or needleless injector systems. The microparticles may also be adapted for administration to mucosa such as the nose, the gastrointestinal tract the colon, vagina or rectum, or administered to the eye.

The microparticles preferably have a size in the range about 10 nm to about 200 μm. The size chosen for a particular microparticle will depend on the active agent to be delivered, and the intended route of administration.

The desired particle size can be obtained by varying the process parameters in manners well known to those skilled in the art. For example changing the particular polymer type used and its molecular weight will change the particle size, an increase in polymer molecular weight generally increasing the particle size. Increasing the polymer concentration may also increase particle size.

The peptide component generally comprises a sequence of amino acids which are designed to bind a receptor on the surface of a cell. Many cell surface receptor binding sequences are known to those skilled in the art. However, as an example, the present invention will generally be described with reference to the Apo B or LDL receptor found on many cells, but this should not be construed as limiting.

Peptide components for use in forming microparticles of the invention contain at least one hydrophobic substituent or moiety capable of acting as an “anchor” for anchoring the peptides to the polymer shell. Hydrophobic moieties or substituents may be derived from biologically compatible hydrophobic compounds such as cholesterol, retinoic acid, C₁₀-C₂₂ fatty acids such as stearic acid (C₁₈) and the like. Further examples of hydrophobic substituents include the following compounds or derivatives thereof which may be attached to the N- and/or C-terminus of the peptide component: Lipid soluble cytotoxic drugs, e.g. etoposide and methotrexate diester; pyrenes or compounds derived therefrom e.g. pyrene butyric acid, benzo(a)pyrene, 3-hydroxybenzo(a)pyrene and benzo(a)pyrene-7,8-dihydrodiol; retinyl derived compounds e.g. N-retinoyl-L-leucyl DOX-14-linoleate; polyunsaturated compounds, e.g. β-carotene; hormones e.g. estradiol, testosterone and aldosterone and the like; diphenylhydantoin; bishydroxycoumarin; pentobarbital; perfluorinated cholesteryl oleate; anthracycline AD-32; PCMA cholesteryl oleate.

These and other suitable hydrophobic compounds are described in Chapter 4 Lipoproteins and Microemulsions as Carriers of Therapeutic and Chemical Agents by Florence & Halbert in the book Lipoproteins as Carriers of Pharmacological Agents Ed. J. Michael Shaw, Publisher Marcel Dekker, Inc., which is incorporated herein by reference in its entirety.

The hydrophobic moiety/substituent can be placed in contact with for example the amino and/or carboxy terminus of the peptide via chemical means such as covalent bonding known in the art. The man skilled in the art will appreciate that peptides of the invention can be assembled using standard Fmc protocols of the Merrifield solid phase synthesis method. The hydrophobic substituent, such as retinoic acid can be activated and attached to, for example, the peptide N-terminus using a standard peptide coupling cycle. For example, initially an acid labile linker such as 3-methoxy-4-hydroxymethylphenoxyacetic acid may be attached to the resin support and esterified with the first amino acid (C-terminus) of the target peptide. When peptide assembly is complete the ester to the linker can be hydrolysed, allowing removal of the fully protected peptide, for example with trifluoroacetic acid (TFA) e.g. 1% TFA, in dichloromethane which can subsequently be evaporated off. At such a stage, the available functional group is the peptide carboxyl, which can be activated with for example one equivalent of dicyclohexylcarbodiimide (DCC) in dimethylformamide (DMF) and coupled to a lipophilic molecule, such as cholesterol (10 equiv), to yield ester. Evaporation of the solvent and treatment with TFA, e.g. 95% TFA, deprotects the amino acid side chains, completing the synthesis. The complete peptide can the be concentrated and precipitated with, for example, diethyl ether to give a solid which can then be washed as necessary to remove any remaining protecting group fragments and excess cholesterol.

N-terminal modifications, such as retinoic acid pyrene butyric acid and stearate addition, targeted at primary amines can be used in the synthesis of modified peptides of the invention using techniques known in the art.

Preferably, peptides capable of being utilised in the invention are amphiphatic in nature, i.e. possess hydrophobic and hydrophilic groups. Suitable hydrophilic groups including hydroxyl, carboxylic and amino groups. Where the peptides are amphipathic in character, the hydrophobic group and hydrophilic groups may be located at any suitable point thereon via appropriate side chains. Preferably the hydrophobic groups and hydrophilic groups are located either at the amino terminus and carboxy terminus of the peptide respectively or vice versa.

The amino acid sequence which makes up the peptides capable of being anchored to the polymer shell of the present invention can be selected from the group of amino acids having basic side chains e.g. lysine, arginine and histidine; amino acids having aliphatic side chains e.g. glycine, alanine, valine, leucine and isoleucine; amino acids having aliphatic hydroxyl side chains e.g. serine and threonine, and derivatives thereof.

Where the binding region amino acid sequence is substantially dissimilar to the binding region sequence of the receptor such as Apo B with respect to the order of amino acids incorporated thereinto, the amino acids selected for inclusion into the binding region of the amino acid sequence can be selected from substantially the same amino acids as those making up the receptor binding region sequence. Naturally, the skilled addressee will understand that conservative replacement and/or substitutions as herein described may also be made to such binding regions.

Naturally, the skilled addressee will appreciate that such amino acid sequences making up functional peptides or polypeptides suitable for use in the present invention must be receptor competent as defined herein. Thus, synthetic or semi-synthetic peptides and/or polypeptides and analogues thereof capable of binding to receptors such as Apo B are encompassed by the present invention.

In a preferment, the amino acid sequence can comprise either or both of the Apo B binding site sequence(s) depicted below in the same peptide or in the form of dimmers or in different peptides:

(1) Lys Ala Glu Tyr Lys Lys Asn Lys His Arg His;
or
(2) Arg Leu Thr Arg Lys Arg Gly Leu Lys;

and analogues thereof which are capable of binding to the Apo B100 receptor site.

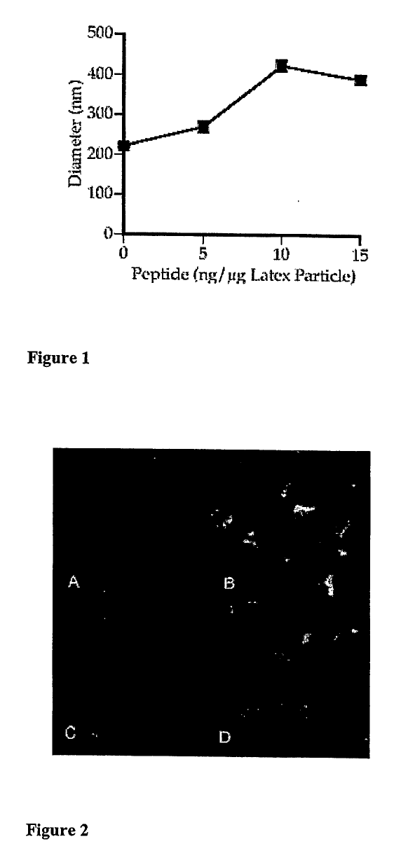

The amino acid sequence can be of any length provided that it is capable of being anchored to the polymer shell under conditions as described herein. The amino acid sequence may include sequences of up to but not including the full length receptor binding protein (e.g. full length Apo B amino acid sequence minus at least one amino acid). Generally however, the amino acid sequence may be up to about 500 amino acid residues long comprising sequences (1) and/or (2) above. Sequences (1) and (2) are known Apo B binding site sequences identified from the human Apo-100 protein as described by Knott T. J. et al Nature Vol. 323 October 1986 p 735. For example, an amino acid sequence could comprise the sequence from amino acid 3079 to about position 3380 of FIG. 1, p 735 (Knott et al supra). The amino acid sequence can comprise at least a single Apo B binding site sequence and can be from about 8-200 amino acid residues in length, or a shorter sequence of from about 8-50 amino acid residues in length, preferably from about 9 to 30 amino acid residues in length. Examples of suitable peptide sequences include those as depicted in Table 1. Naturally, the skilled addressee will appreciate that practical considerations such as the ability of the amino acid sequence to bind to receptor and ability to synthesise the peptide sequence generally means that the shorter amino acid sequences are preferred. The skilled addressee will appreciate that natural variations in the amino acid sequences comprising amino acid substitutions, deletions and/or replacements are encompassed by the present invention. Furthermore, the skilled addressee will also appreciate that amino acid substitutions, deletions and/or replacements can be made to the amino acid sequence so long as such modifications do not substantially interfere with the ability of the amino acid sequence to bind to a binding site and thereby elicit a physiological response. For example, conservative replacements may be made between amino acids within the following groups:
(i) Lysine and arginine;
(ii) Alanine, serine and threonine;
(iii) Glutamine and asparagines;
(iv) Tyrosine, phenylalanine and tryptophan; and
(v) Leucine, isoleucine, valine and methionine.
   so long as the physiological function of the peptide is not substantially impaired.

Typically the amount of peptide to microparticle may be about 5 to 10 ng peptide/μg of microparticle.

The present inventors have observed that the microparticles can cause aggregation of cells, which may be undesirable. Thus, it may also be desirable when adding the microparticles to cells, to include an agent designed to minimise or reduce aggregation of the cells. For example an antibody capable of binding the receptor to which the peptide is designed to bind, may be used.

The particles may be made by techniques similar to those used to form liposomes and niosomes, for instance by blending the components in an organic solvent and then contacting the dried mixture with an aqueous solution, optionally followed by a particle size reduction step see for example EP99907729.0.

In a further aspect there is provided a method of forming a peptide modified polymer microparticle, which comprises:
forming a non-aqueous solution comprising a polymer, and an agent or agents;
   forming a dispersion of an aqueous liquid in the non-aqueous solution;
   sonicating the dispersion so as to form microparticles; and
   evaporating off the non-aqueous solution so as to leave an aqueous liquid comprising the microparticles, wherein the hydrophobically modified peptide may be included in the initial non-aqueous solution, or may be added to the aqueous liquid after microparticle formation.

Preferably an emulsifier is included in the initial aqueous liquid.

Typically the non-aqueous solvent into which the components are dissolved may be dichloromethane.

In a further aspect of the invention there is provided use of peptide modified microparticles of the present invention as a cell targeting vector.

In a still further aspect there is provided use of peptide modified microparticles of the present invention for the manufacture of a medicament for the treatment or prophylaxsis of a disease.

In a further aspect there is provided a pharmaceutical formulation comprising the peptide modified microparticles of the present invention together with a pharmaceutically acceptable carrier. Suitable pharmaceutically acceptable carriers are known to those skilled in the art.

### DETAILED DESCRIPTION OF THE INVENTION

The present invention will now be further described by way of example and with reference to the Figures which show:

FIG. 1 shows a graph of the effect of peptide adsorption on the diameter of polystyrene latex particles. Mean ±standard deviation n=10. Peptide 3 and Sigma carboxylate modified latex particles;

FIG. 2 shows photographs of the effect of temperature on the cellular association of peptide modified latex microparticles on HFFF-2 fibroblast cellsCells incubated overnight with serum free medium and then with test system for 3 hours at 37° C. (A and B) or 4° C. (C and D). A and C control wells with media only, B and D peptide modified latex. Latex system 10 ng Peptide 1/μg Fluoresbrite latex and 0.15 μg latex/μL of media. All windows confocal microscopy;

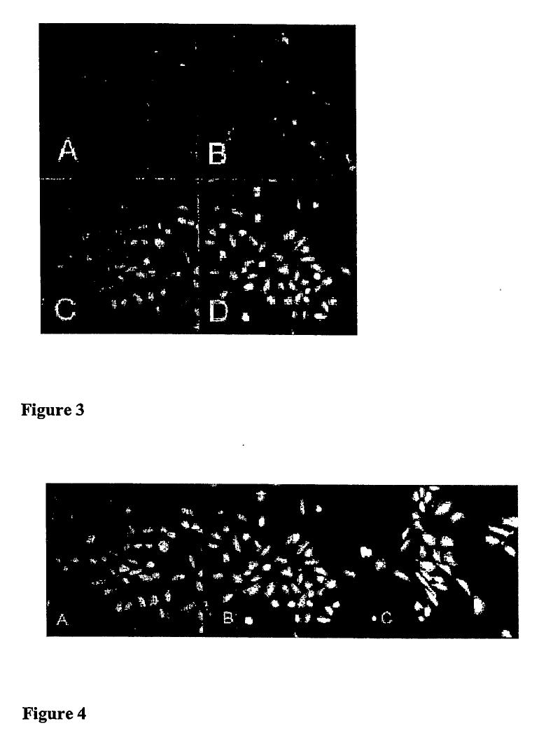

FIG. 3 shows photographs of the effect of time on the cellular association of peptide modified latex microparticles on CHO cells. CHO incubated overnight with serum free medium and then with peptide modified latex 37° C. for 1 hours (A and B) and 3 hours (C and D). Latex system A and C long Peptide 1/μg Sigma carboxylate modified latex, B and D 10 ng Peptide 3/μg Sigma carboxylate modified latex both at 0.15 μg latex/μL of media. All windows confocal microscopy;

FIG. 4 shows photographs of the effect of peptide variation on the cellular association of peptide modified latex microparticles on CHO cells. CHO incubated overnight with serum free medium and then with peptide modified latex 37° C. for 3 hours (A, B and C). Latex system A 10 ng Peptide 1/μg Sigma carboxylate modified latex, B 10 ng Peptide 3/μg Sigma carboxylate modified latex, C 10 ng Peptide 4/μg Sigma carboxylate modified latex all at 0.15 μg latex/μL of media. All windows confocal microscopy;

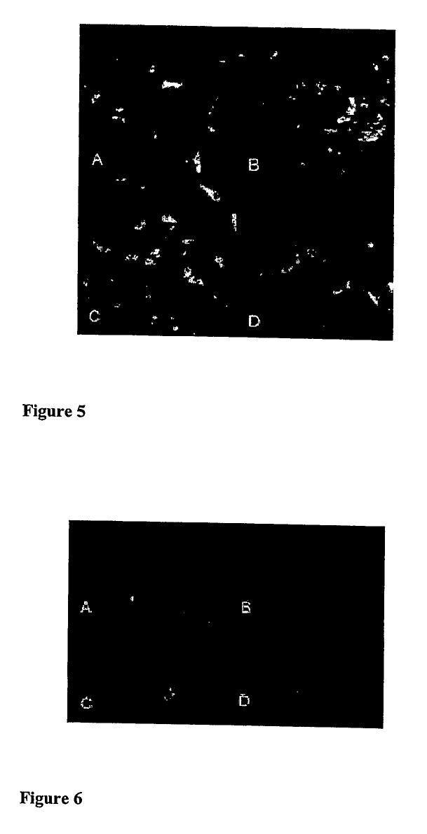

FIG. 5 shows photographs of the effect of anti-LDL receptor antibody on the cellular association of peptide modified latex microparticles of HFFF-2 fibroblast cells. Cells incubated overnight with serum free medium and then with test system for 3 hours at 37° C. A and C wells with peptide modified latex only, B and D peptide modified latex plus anti-LDL receptor antibody at 8 μg/mL. Latex system A and B long Peptide 1/μg Fluoresbrite latex, C and D long Peptide 4/μg Fluoresbrite latex both at 0.15 μg latex/μL of media. All windows confocal microscopy; and

FIG. 6 shows further photographs of the effect of anti-LDL receptor antibody on the cellular association of peptide modified latex microparticles on HFFF-2 fibroblast cells. Cells incubated overnight with serum free medium and then with test system for 3 hours at 37° C. A and C wells with peptide modified latex only, B and D peptide modified latex plus anti-LDL receptor antibody at 8 μg/mL. Latex system A and B long Peptide 1/μg Sigma carboxylate modifed latex, C and D long Peptide 3/μg Sigma carboxylate modified latex both at 0.15 μg latex/μL of media. All windows normal fluorescent microscopy.

Materials and Methods

Polystyrene Latex

Two Latex particles were employed.

Polystyrene carboxylate modified, Sigma (L5155) diameter 32 nm, concentration 2.5% w/v.

Fluoresbrite YG plain latex microspheres, Polysciences, diameter 64 nm, concentration 2.5% w/v.

Peptides

Peptides were obtained from Thistle Peptides, Glasgow at 95% purity and used as received. Chemical structures of the individual peptides are presented in Table 1.

TABLE 1

| Pep- | | | |
|---|---|---|---|
| tide | N-terminal | Sequence | C-terminal |
| 1 | Retinoic | Leu-Arg-Leu-Thr-Arg- | Cholesterol |
| | Acid | Lys-Arg-Gly-Leu-Lys- | |
| | | Leu- | |
| 2 | Retinoic | Gly-Thr-Thr-Arg-Leu- | -COOH |
| | Acid | Thr-Arg-Lys-Arg-Gly- | |
| | | Leu-Lys-Leu- | |
| 3 | Retinoic | Tyr-Lys-Leu-Glu-Gly- | Cholesterol |
| | Acid | Thr-Thr-Arg-Leu-Thr- | |
| | | Arg-Lys-Arg-Gly-Leu- | |
| | | Lys-Leu-Ala-Thr-Ala- | |
| | | Leu-Ser- | |
| 4 | Pyrene | Lys-Leu-Glu-Gly-Thr- | Cholesterol |
| | Butyric | Thr-Arg-Leu-Thr-Arg- | |
| | Acid | Lys-Arg-Gly-Leu-Lys- | |
| | | Leu-Ala-Thr-Ala-Leu- | |
| | | Ser-Leu-Phe-Leu-Phe | |

Peptide Solutions

Each peptide (5 mg) was dissolved in DMSO and made to a final volume of 2 ml. This solution was employed in the experiments detailed below and stored at −20° C.

Peptide Attachment

Latex (50 to 100 μL) was added to PBS (890 to 945 μL) followed by peptide solution (5 to 10 μL) and the resultant 1 mL solution mixed. The material was left to stand at room temperature for 2 hours and then dialysed over night against 2 L of PBS in the dark at 4° C. The particles were then recovered stored at 4° C. in the dark and used without further treatment.

CHO-K1 (ECACC Number 85051005)

CHO stock culture was grown in Ham's F12 media supplemented with 10% foetal bovine serum, glutamine (2 mM), fungizone (50 mg/ml) and pen-strep (0.1 mg/ml). Cells were seeded at 1 to 2×10⁴ cell/cm² using 0.25% trypsin-EDTA and maintained in a humidified 5% CO₂ atmosphere, at 37° C. and sub-cultured twice a week.

HFFF2 (ECACC Number 86031405)

HFFF2 stock culture was grown in Dulbecco's modified Eagle's media supplemented with 10% v/v foetal bovine serum, glutamine (2 mM), fungizone (50 mg/ml) and pen-strep (0.1 mg/ml). Cells were seeded at 2 to 3×10⁴ cell/cm² using 0.25% trypsin-EDTA and maintained in a humidified 5% CO₂ atmosphere, at 37° C. and sub-cultured twice a week.

Cellular Uptake Assays

Fluorescence Microscopy

Prior (48 hr) to an experiment CHO or HFFF2 cells were plated at 2×10⁴/cm² in an 8 well chamber slide, 0.7 cm² and 0.2 mL/chamber. One day before the experiment, media was replaced with a similar media but containing no lipids. On the date of the experiment, the cells for 4° C. incubation were chilled for 15 minutes prior to any additions. The wells were washed with 0.1 mL of phosphate buffered saline (PBS) and the synthetic LDL microparticle system, in media, added. The slides were then incubated for 1 or 3 hours at either 4° C. or 37° C. the latter in a humidified 5% CO₂ atmosphere. After incubation, the cells were washed twice with PBS and fixed with 0.02 mL of 2% glutaraldehyde in PBS for 15 minutes at 4° C. The cells were finally washed twice with PBS and the slides visualized in fluorescent microscope (Reichert-Jung Polyvar).

Confocal Microscopy

Prior (48 hr) to an experiment CHO or HFFF2 cells were plated at 2×10⁴/cm² in an 8 well chamber slide, 0.7 cm² and 0.2 mL/chamber. One day before the experiment, media was replaced with a similar media but containing no lipids. On the day of the experiment, the cells for 4° C. incubation were chilled for 15 minutes prior to any additions. The wells were washed with 0.1 mL of phosphate buffered saline (PBS) and the synthetic LDL system in media added. The slides were then incubated for 1 or 3 hours at either 4° C. or 37° C. the latter in a humidified 5% CO₂ atmosphere. After incubation, the cells were washed twice with PBS and fixed with 0.02 mL of 2% glutaraldehyde in PBS for 15 minutes at 4° C. The cells were finally washed twice with PBS and then visualized in a confocal microscope (Bio-Rad 1024, Bio-Rad) using a Krypton-Argon laser at an excitation wavelength of 488 nm with emission collected using a 525 nm (+/−25 nm) filter. The lens employed was ×20 PA with a numerical aperture of 0.75 and the data analysed using “Laser-sharp” software (Bio-Rad).

Results

The interaction of the peptide with latex particles is presented in FIG. 1, which illustrates that increasing the peptide coverage increases particle diameter. Without being bound by theory this effect is probably related to adsorption of the peptide onto the latex leading to colloidal de-stabilisation and aggregation. In order to minimise this effect surface coverage around 5 to 10 ng peptide/μg of latex particle may be preferred.

Confocal Microscopy

Confocal microscopy was employed to examine the interaction of the peptide coated latex particles with either HFFF-2 fibroblast cells or Chinese Hamster Ovary (CHO) cells in tissue culture. In FIG. 2 a blank preparation of CHO cells is shown both in normal illumination and in confocal microscopy. The latter indicates that the cells exhibit a degree of autofluorescence even without exposure to the latex particles.

Fibroblast cells after exposure to the peptide coated latex particles exhibit an increased cellular association demonstrated in FIG. 2 by an increased fluorescence in panels B and D. However, the association at 37° C. (panel B) is obviously greater than at 4° C. (panel D) indicating that the association is a temperature dependent phenomenon. The increased fluorescence at 4° C. is indicative of non-specific binding which is present in all lipoprotein cell binding experiments.

If the binding experiment is assessed over time the cell associated fluorescence increases with time, FIG. 3. The fluorescence at 1 hour (panel A and B) is lower than 3 hours, indicating that the association is a time dependent process. In addition FIG. 3 present the differences between the peptides employed in the experiment. There does not appear to be any difference between either peptide at 1 hour and potentially a difference at 3 hour. This is enforced by the results presented in FIG. 4 after 3 hours incubation, where the peptide effect is demonstrated with an increased cellular association for peptide 4 followed by peptide 3 and then peptide 1. The results indicate that the degree of cellular association is peptide dependent.

The addition of an antibody which binds to the LDL receptor should reduce the cellular association of the peptide modified latex particles. The results for these experiments are presented in FIGS. 5 and 6. The results for peptide 1 (panel A and B) indicate that the antibody does decrease the cellular association of the latex. With peptide 4 (panel C and D) the reduction is not as great. If the experiment is performed using normal fluorescent microscopy (FIG. 7) the reduction due to the presence of the antibody is clearly visible for both peptide 1 and 3. These results indicate that the anti-LDL receptor antibody acts to reduce peptide modified latex cellular association indicating that this association arises via and interaction with the LDL receptor.

### Conclusions

The peptides used in the preparation of sLDL will interact with latex microparticles and adsorb onto the particle's surface. The peptide treated particles interact with both fibroblast and CHO cells in a time and temperature dependent manner, the latter indicating uptake via an energy dependent process. The degree of interaction is controlled by the structure of the peptide employed to coat the microparticles, indicating a degree of structural specificity. Finally, the inclusion of an anti-LDL receptor antibody reduces the degree of cellular association.

The results indicate that latex particles surface modified with sLDL peptides interact with cells via a receptor dependent mechanism most probably the LDL receptor. This route will therefore be useful for the delivery of any suitable agent to cells via the LDL receptor.

1. A microparticle for use in delivering an agent or agents to a cell, the microparticle comprising:
a) a polymer shell;
b) an agent or agents for delivery to a cell; and
c) a peptide component comprising a hydrophobic moiety wherein the hydrophobic moiety is capable of anchoring the peptide to the polymer shell and the peptide is intended to target the microparticle to a receptor on the surface of the cell.

2. The microparticle according to claim 1 wherein the polymer shell is biodegradable and/or biocompatible.

3. The microparticle according to claim 1 wherein the polymer shell is made from polyesters such as polylactide, polyglycolide, copolymers of lactide and glycolide, polyhydroxybutyrate, polycaprolactone, copolymers of lactic acid and lactone, copolymers of lactic acid and PEG, copolymers of a-hydroxy acids and α-amino acids(polydepsipeptides), polyanhydrides, polyorthoesters, polyphosphazenes, copolymers of hydroxybutyrate and hydroxyvalerate, poly(ethylene carbonate), copoly(ethylene carbonate), polyethylene terephthalate, polystyrene/latex polymers, or mixtures of these polymers.

4. The microparticle according to claim 1 wherein the microparticle has a size of 10 nm to 200 μm in diameter.

5. The microparticle according to claim 1 wherein the agent is a therapeutic, pharmaceutical, pharmacological, diagnostic, cosmetic, prophylatic, herbicidal, pesticidal and/or fertilizer agent.

6. The microparticle according to claim 5 wherein the agent is an antigen.

7. The microparticle according to claim 1 for use in delivery of the agent to cells which are in vitro or in vivo.

8. The microparticle according to claim 1 which has been adapted to be administered by injection, topically or mucosally.

9. The microparticle according to claim 1 wherein the hydrophobic moiety is derived from cholesterol, retinoic acid, C₁₀-C₂₂ fatty acids such as stearic acid (C₁₈) and the like.

10. The microparticle according to claim 1 wherein the hydrophobic moiety is a lipid soluble cytotoxic drugs, e.g. etoposide and methotrexate diester; pyrenes or compounds derived therefrom e.g. pyrene butyric acid, benzo(a)pyrene, 3-hydroxybenzo(a)pyrene and benzo(a)pyrene-7,8-dihydrodiol; retinyl derived compounds e.g. N-retinoyl-L-leucyl DOX-14-linoleate; polyunsaturated compounds, e.g. β-carotene; hormones e.g. estradiol, testosterone and aldosterone and the like; diphenylhydantoin; bishydroxycoumarin; pentobarbital; perfluorinated cholesteryl oleate; anthracycline AD-32; PCMA cholesteryl oleate.

11. The microparticle according to claim 1 wherein the peptide is intended to target the ApoB receptor.

12. The microparticle according to claim 11 wherein the peptide component designed to bind to the ApoB receptor comprises either or both of the Apo B binding site sequence(s) depicted below in the same peptide or in the form of dimers or in different peptides:
(1) Lys Ala Glu Tyr Lys Lys Asn Lys His Arg His; or
(2) Arg Leu Thr Arg Lys Arg Gly Leu Lys;
and analogues thereof which are capable of binding to the Apo B100 receptor site.

13. A formulation comprising a microparticle according to claim 1 for use in delivering an agent to a cell.

14. The formulation according to claim 13 wherein the formulation is a pharmaceutical formulation and optionally comprises a pharmaceutical carrier therefore.

15. The formulation according to claim 13 further comprising an agent designed to minimise or reduce aggregation of cells.

16-17. (canceled)

18. A method of forming a peptide modified polymer microparticle, which comprises:
a) forming a non-aqueous solution comprising a polymer, and an agent or agents;
b) forming a dispersion of an aqueous liquid in the non-aqueous solution; sonicating the dispersion so as to form microparticles; and
c) evaporating off the non-aqueous solution so as to leave an aqueous liquid comprising the microparticles, wherein the hydrophobically modified peptide may be included in the initial non-aqueous solution, or may be added to the aqueous liquid after microparticle formation.

19. The method according to claim 18 wherein an emulsifier is included in the initial aqueous liquid.
